(19) United States
(12) Patent Application Publication
(10) Pub. No.: US 2004/0013733 A1
(43) Pub. Date: Jan. 22, 2004

(54) Preparation of a biodegradable thermal-sensitive gel system

(75) Inventors: Yuan-Han Chen, Hsinchu (TW); Ming-Hsi Yeh, Chiai (TW); Huey-Min Lai, Hsinchu (TW)

Correspondence Address: BACON & THOMAS, PLLC, 625 SLATERS LANE, FOURTH FLOOR, ALEXANDRIA, VA, 22314

(73) Assignee: Industrial Technology Research Institute, Hsinchu (TW)

(21) Appl. No.: 10/330,085

(22) Filed: Dec. 30, 2002

(30) Foreign Application Priority Data
Dec. 28, 2001 (TW) 90132965
Oct. 21, 2002 (TW) 91124213

(51) Int. Cl. A61K 38/39; A61K 31/737; A61K 31/716; A61K 9/14

(52) U.S. Cl. 424/488; 514/2; 514/54; 514/55; 514/57

(57) ABSTRACT

The present invention relates to a biodegradable thermal-sensitive gel system, which comprises at least one polysaccharide solution, at least one electrolytic salt, and at least one buffer solution for adjusting pH. A natural protein as well as a cross-linking agent can be added to the gel system optionally. Said gel system is liquid at room temperature (25° C.) and solidifies at or above 37° C.

The present invention also relates to a process for preparing said gel system, and a use for drug releasing carrier.

### BACKGROUND OF THE INVENTION

1. Field of the Invention

The present invention relates to a thermal-sensitive gel system and the process for preparing the same and, more particularly, to a biodegradable thermal-sensitive gel system and it's preparing process.

The present invention further relates to the application of said gel system.

2. Description of Related Art

In recent years, scientists focus on the development and design of sensitive intellectual materials which response promptly to the environmental factors such as temperature, pH value, electric current, electric field or radiation. A main purpose to develop such kind of polymeric material is to apply in drug delivery, tissue engineering, and biotechnology. For example, poly(N-isopropylacrylamide) (PNIPAAM) is a well-known material used as a thermal-sensitive gel system for the purpose illustrated above. When the temperature is below 32° C., the LCST (Lower Critical Solution Temperature) of PNIPAAM, it dissolves in water completely and become a transparent solution. On the other hand, it solidifies at a temperature higher than 32° C. The prior arts related to PNIPAAM have been disclosed in U.S. Pat. No. 6,238,688 wherein Wu. et al proposed a thermal-sensitive composite material, which served as a blood vessel repairing system and drug delivery system. The hydrophobic PNIPAAM was blended with little amount of hydrophilic protein (eg. Gelatin) to adjust the strength and softness of the gel. In addition, in U.S. Pat. No. 6,194,073, Li et al disclosed a gel system consisting of at least two polymers, such as polyacryamide and PNIPAAM, while the former is a gel stable to environment and the other is unstable to the environment. The volume of the gel varies with the environmental factor such as temperature.

PNIPAAM, however, is characterized by severe dehydration phenomenon at 37° C., and will generate some problems for applying to tissue engineering. Moreover, PNIPAAM is toxic and non-biodegradable, thus the biological compatibility of PNIPAAM is very poor for biomedical use.

Besides, Jeong et al has published a novel thermal-sensitive material, Polyethylene oxide(PEO)/polylactide (PLA) co-polymer (Jeong, B., Y. H. Bae, D. S. Lee, and S. W. Kim, Nature, 388, p860, 1997), whose physical state in solution depends on the temperature range. The material is in liquid state at 45° C., and becomes semi-solid state as temperature decreases. The material disclosed by Jeong et al is biodegradable and thermal-sensitive. However, it won't become liquid unless the temperature is higher than 45° C., which is too hot for human body and thus not suitable for administrating to the tissue required repairing. This disadvantage also makes it not proper for encapsulating bio-active materials such as drug or cells since they will lose their activity or be injured under such high temperature.

Poly(ethylene oxide)-poly(propylene oxide)-poly(propylene oxide) bulk co-polymer is also used as a thermal-sensitive polymeric material. However, but the high effective concentration, corrosive and non-biodegradable property, as well as allergic response inducing factor makes it improper for biomedical use.

Therefore, it needs to develop a novel material which is low toxic, biodegradable, high biological compatibility to improve the aforementioned disadvantages of the well-known polymers.

### SUMMARY OF THE INVENTION

The main object of the present invention is to provide a biodegradable thermal-sensitive gel system which is neutral, low toxic, and biodegradable. The biological compatibility of gel system is so excellent that is proper for applying to various biomedical fields such as drug delivery and tissue engineering.

Another object of the present invention is to provide a process for preparing said biodegradable thermal-sensitive gel system. The solidification time and viscosity of said gel system can be simply adjusted by the concentration of electrolytic salts and cross-linking condition.

One aspect of the present invention is to provide a gel system comprising collagen/polysaccharide composite, with an extracellular-like matrix structure suitable for tissue cell attachment and growth. In addition, said gel system contains many hydrophilic functional groups which are able to capture plenty of water molecules to avoid dehydration. So it is excellent for filling up or repairing injured tissue.

Another aspect of the present invention is to provide a drug carrying vector which is biologically compatible and biodegradable. Said vector maintains the activity of drugs and is easy to be administrated.

Another aspect of the present invention is to provide a drug (eg. angiogenesis drug) delivery system which contains a gel with porous structure to form a delivery channel and release drug well.

Another aspect of the present invention is to provide a angiogenesis method which administers drug directly to where requires it and provides a scaffold to support blood vessel growth, the scaffold will degrade and adsorbed automatically, so taking-out by operation is not needed.

To achieve the object, the biodegradable thermal-sensitive gel system of the present invention includes at least one polysaccharide solution, at least one electrolytic salt, and at least one weak alkaline solution.

The present invention also relates to a process for preparing a biodegradable thermal-sensitive gel system, comprising mixing a polysaccharide solution and electrolytic salt to form a mixture, subsequently adding a weak alkaline solution to adjust the pH of said mixture.

The present invention also relates to a drug delivery system for angiogenesis, comprising a biodegradable thermal-sensitive gel system which contains at least one polysaccharide solution, at least one electrolytic salt, and at least one weak alkaline solution.

The present invention also relates to an angiogenesis method, comprising co-administrating an angiogenesis drug, at least one polysaccharide solution, at least one electrolytic salt, and at least one weak alkaline solution to the tissue requiring angiogenesis.

Other objects, advantages, and novel features of the invention will become more apparent from the following detailed description when taken in conjunction with the accompanying drawings.

### BRIEF DESCRIPTION OF THE DRAWINGS

FIG. 1 is a flow chart of the gel system preparing process of the present invention;

FIG. 2 shows the growth state of blood vessel observed by SEM;

FIG. 2a shows the site tissue taken out from;

FIGS. 2b to 2d shows the blood growth state after i.s. injection of growing factor for 14 days observed by SEM; wherein FIG. 2b shows the blood vessels growth state of the interface between gel and subcutaneous tissue, 200×; FIG. 2c shows the blood vessels growth state of connective tissue, 1000×; FIG. 2d shows the blood vessels growth state of connective tissue, 2000×;

FIGS. 2e to 2g shows the blood vessels growth state after i.s. injection of growing factor and S-1-P for 14 days by SEM; wherein FIG. 2e shows the blood vessels growth state in the gel, 200×; FIG. 2f shows the blood vessels growth state in the gel, 1000×; FIG. 2g shows the blood vessels growth state in the gel, 2000×;

FIG. 3 is an electron microscope picture showing the porous structure of the gel of the present invention.

### DETAILED DESCRIPTION OF THE PREFERRED EMBODIMENT

The present invention relates to a biodegradable thermal-sensitive gel system, comprising at least one polysaccharide solution, at least one electrolytic salt, and at least one weak alkaline solution for adjusting the pH value of the gel system until it is almost neutralized. Preferably, said polysaccharide solution is neutral or acidic natural polysaccharide solution, and said polysaccharide is selected from the naturally hydrophilic polysaccharide group consisting of chitin, chitosan, glycosaminoglycan, and cellulose, wherein chitin or chitosan is more preferred. Said neutral or acidic solution is preferred pharmaceutically acceptable neutral or acidic solution, and the neutral solution is preferred water-soluble salt solution while acidic solution is preferred acetic acid solution, lactic acid solution, citric acid solution, and diluted hydrochloric acid solution. Said weak alkaline solution is preferred sodium bicarbonate, potassium bicarbonate and phosphate buffer saline (PBS). The pH value of said gel system is preferred between pH 6.0 and 8.0. The concentration of said electrolytic salt solution is preferred between 2.0 wt % and 12.0 wt %, and said salt is preferred selected from the group consisting of glycerol-phosphate, sorbitol-phosphate and glucose-phosphate.

The biodegradable thermal-sensitive gel system of the present invention can further comprise a natural protein to enhance the softness of the gel. Said protein is preferred selected from the natural hydrophilic protein group consisting of gelatin, collagen, fibrin, fibronectin, and elastin, and collagen is more preferred. The weight ratio of said natural protein to said polysaccharide ranges from 1:20 to 1:3; preferably, 1:36 to 1:6.

The biodegradable thermal-sensitive gel system of the present invention can further comprise a cross-linking agent to control the solidification time. Preferably, said cross-linking agent is glutaldehyde(GA), 1,4-butanediol diglycidyl ether(BDDGE), 1-ethyl-3-(3-dimethyl aminopropyl) carbodiimide (EDC), or Genipin. The concentration of said cross-linking agent preferably ranges from 0.01 wt % to 0.2 wt %, and the cross-linking reaction is preferably carries out in a weak acidic or a weak alkaline solution.

The present invention also relates to a process for preparing a biodegradable thermal-sensitive gel system, comprising mixing a polysaccharide solution and electrolytic salt to form a mixture, subsequently adding a weak alkaline solution to adjust the pH value of said mixture. The process further comprises a step of adding a natural protein to said gel system wherein the weight ratio of said natural protein to said polysaccharide is between 1:36 w/w and 1:6 w/w. It can further comprises a step of adding a cross-linking agent to said gel system to control the solidification time and the viscosity of the gel. Said polysaccharide solution, electrolytic salts, weak alkaline solution, natural protein and cross-linking agent is the same as those described above.

Furthermore, the gel system of the present invention can be adjusted to a neutral solution before solidification, and thus can be easily mixed with drugs before administration. The gel is non-toxic and conforms to the neutral environment in human body. Besides, the formed gel has porous structure, which allows the drug inside being released out steadily. Therefore, the gel could serve as an excellent drug matrix and delivery system.

The present invention also provides an angiogenesis method which co-administrates the gel system of the present invention and angiogenesis-inducing drug to the tissue required for angiogenesis. In one embodiment of the present invention, said gel system was mixed with bFGF and S-1-P and subcutaneous injected to mammals. The temperature of mammal's body is 37° C., so that the gel system will solidify and encapsulate the drug under this circumstance, and followed by releasing drug steadily to maintain the potency. The solid gel here is also porous and strong enough to provide an excellent support for angiogenesis.

The present invention exploits some natural materials such as collagen and chitosan, electrolytic salts and cross-linking agent to prepare a biodegradable thermal-sensitive gel, which is in liquid phase at room temperature (25° C.) and starts to solidify at 37° C. The preparing method of the present invention comprises a crucial step to adjust the pH value of the gel system by adding a weak alkaline solution such as PBS to fit in with the neutral pH value in human body. After solidification, the gel has extracellular-like structure which is apt to cell attachment or other biomedical applications.

The details of the present invention are illustrated by following preferred embodiments.

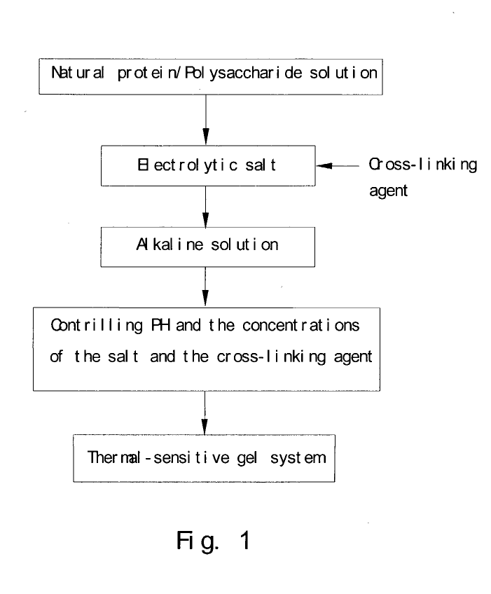

The preparation process of the present invention is as the flow chart of FIG. 1.

### EXAMPLE 1

### The Gel System Without Natural Proteins

5 mL of 4 wt % chitosan (in 1 wt % acetic acid) was added into 3 ML of PBS (pH 7.6) at room temperature with stirring. 1 ml of 56 wt % glycerol-phosphate was then added to the mixture followed by adding 1 mL of 0.5 M NaHCO₃ to adjust the pH value of the solution to 7.2. The PBS and sodium bicarbonate are used to adjust the pH value of the gel system. The product thus obtained is liquid and will solidify while the temperature rises to 37° C., which needs about 3 minutes.

### EXAMPLE 2

### The Gel System Containing Natural Proteins

4 mL of 4 wt % chitosan (in 1 wt % acetic acid) and 1 mL of 1 wt % collagen (in 1 wt % acetic acid) were added into 3 mL of PBS (pH 7.6) at room temperature with stirring. 1 ml of 56 wt % glycerol-phosphate was then added to the mixture followed by adding 1 mL of 0.5 M NaHCO₃ to adjust the pH value of the solution to 7.2. The product thus obtained is liquid and will solidify while the temperature rises to 37° C., which needs about 3 minutes.

### EXAMPLE 3

### The Gel System Containing Different Salt Concentration

4 mL of 4 wt % chitosan (in 1 wt % acetic acid) and 1 mL of 1 wt % collagen (in 1 wt % acetic acid) were added into 3 mL of PBS (pH 7.6) at room temperature with stirring. 2 ml of 56 wt % glycerol-phosphate was then added to the mixture followed by adding 1 mL of 0.5 M NaHCO₃ to adjust the pH value of the solution to 7.2. The product thus obtained is liquid and will solidify while the temperature rises to 37° C., which needs about 3 minutes.

### EXAMPLE 4

### The Gel System Containing Cross-Linking Agents

4 mL of 4 wt % chitosan (in 1 wt % acetic acid) and 1 mL of 1 wt % collagen (in 1 wt % acetic acid) were added into 2.9 mL of PBS (pH 7.6) at room temperature with stirring. 1 ml of 56 wt % glycerol-phosphate was then added to the mixture followed by adding 1 mL of 0.5 M NaHCO₃ to adjust the pH value of the solution to 7.2. The mixture was subsequently added by 0.1 mL of 10 wt % BDDGE (in PBS) and agitated well until a clear solution performs. The product thus obtained is liquid and will solidify while the temperature rises to 37° C., which needs about 2 minutes.

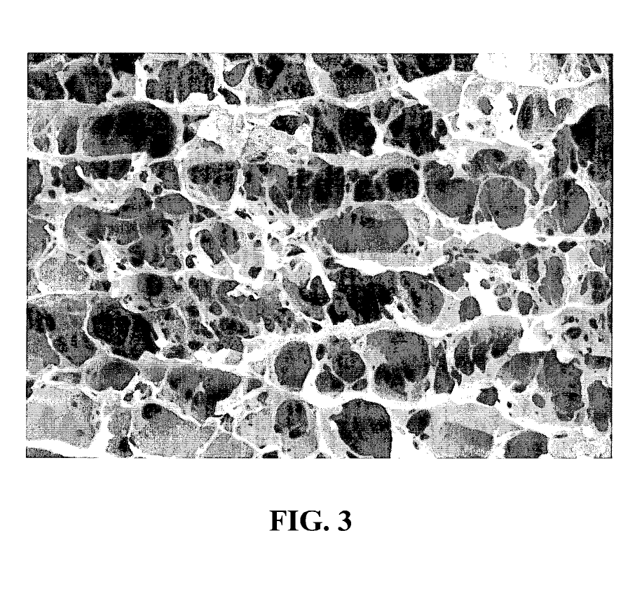

With comparison of example 1, addition of cross-linking agents will shorten the solidification time and improve the mechanical strength of the gel. The structure of said solid gel is shown in FIG. 3, with porous beehive structure.

### EXAMPLE 5

### The Angiogenesis Animal Model

The gel solution prepared according the process of Example 4 was mixed with various drugs listed in tablet at room temperature and is injected (0.5 mL gel each) to two months old mice, N=2. Sacrifice the mice after 15 days and take out the gel and its adjacent tissue to observe the blood vessel growth. The results are listed in table 1 and shown in the color pictures supplemented (please refer to attachments).

TABLE 1

| Group | Drug | Blood vessel growth state |
|---|---|---|
| Blank control | PBS | Angiogenesis is not obvious |
| group | | (as attachment 1). |
| Group 1 | bFGF (250 ng/ml) | Angiogenesis is present |
| | | (as attachment 2). |
| Group 2 | bFGF(250 ng/ml) + | Angiogenesis is apparent. |
| | S-1-P(10 μg/ml) | (As attachment 3). |

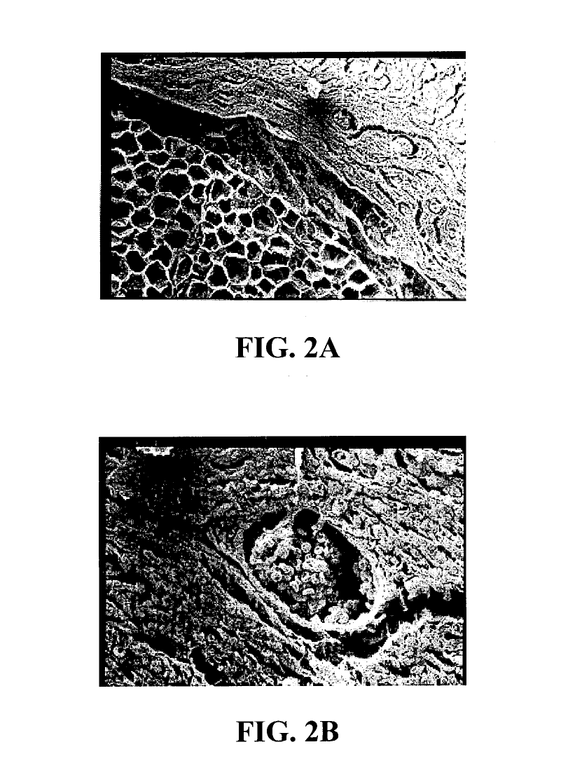

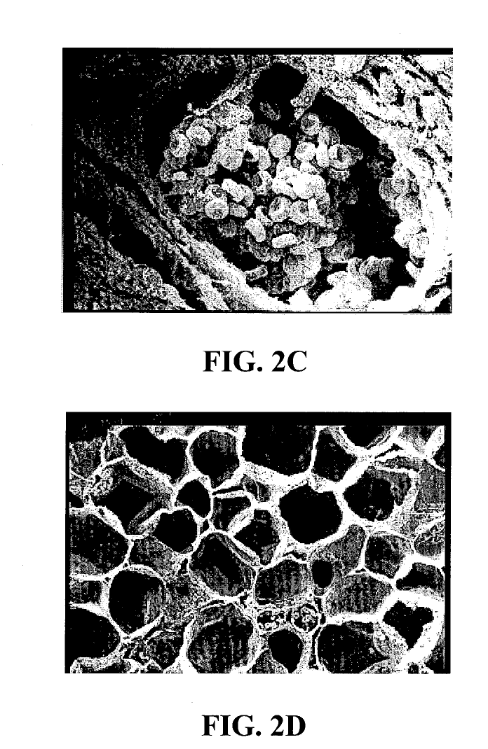

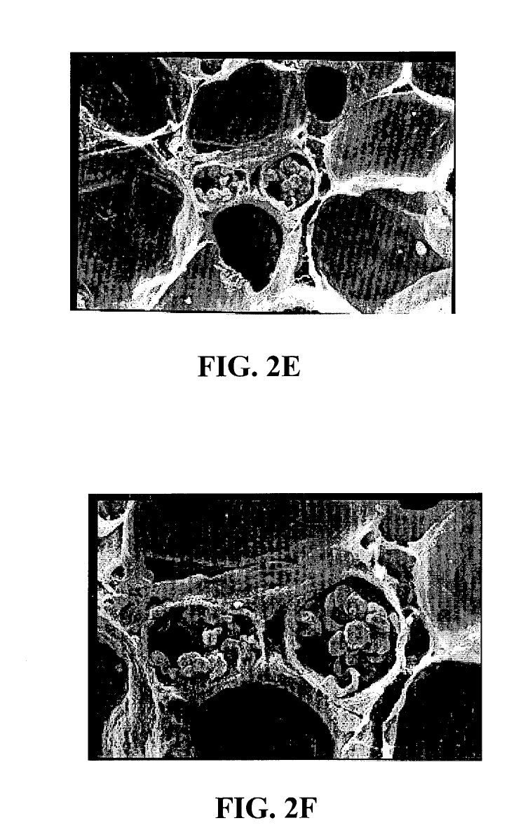

The tissue taken out was observed by electron microscope and the results were shown as FIG. 2. FIG. 2a shows the site tissue taken out from; FIGS. 2b to 2d shows the blood vessels growth state after i.s. injection of growing factor for 14 days by SEM. FIG. 2b shows the blood vessels growth state of the interface between gel and subcutaneous tissue, 200×; the dense part shown in the right-up side of the figure is the connective tissue while the beehive structure shown in the left-down part is the gel of the present invention. FIG. 2c shows the blood vessels growth state of connective tissue, 1000×; where erythrocytes present is where blood vessels pass though. FIG. 2d shows the blood vessels growth state of connective tissue, 2000×;

FIGS. 2e to 2g shows the blood vessels growth state after i.s. injection of growing factor and S-1-P for 14 days observed by SEM. FIG. 2e shows the blood vessels growth state in the gel, 200×; FIG. 2f shows the blood vessels growth state in the gel, 1000×; FIG. 2g shows the blood vessels growth state in the gel, 2000×. The figures obviously show that plenty of erythrocytes present in the beehive structure of the gel, which indicates angiogenesis occurring in the gel. In other words, the endothecium cell and blood vessels of the host can develop in the beehive structure of the gel. The results tell us that co-administrate the gel of the present invention and angiogenesis drug did benefit angiogenesis.

The biodegradable thermal-sensitive gel system of the present invention can provide a matrix having a structure like cells, a matrix for the attachment of cell receptors or cells, an alternative path or rate of dissociation by assistance of enzymes, and to extend the time for dissociation. Therefore, the biodegradable thermal-sensitive gel system of the present invention is suitable for the attachment of cells or used for the culture of cells.

On the other hand, the viscosity of the biodegradable thermal-sensitive gel system of the present invention can be controlled by adjusting the concentration of the electrolyte and the condition of the cross-linking. The hydrophility of the hydrophobicity of the surface of the biodegradable thermal-sensitive gel system of the present invention can be easily modified through adjusting the percentages of the components. Therefore, the biodegradable thermal-sensitive gel system of the present invention can be also applied to drug delivery, cell culture or tissue engineering.

From the descriptions illustrated, it can be realized that the gel system of the present invention did provide an excellent environment for angiogenesis. The blood vessels will grow into the gel if we administrate the gel system to injured tissue, thus preventing from severe injure. One potential application is to administrate the gel and angiogenesis drug to diabetics who suffers from low limb tissue necrosis. The solid gel could replace the injured tissue to support the blood vessels, and the angiogenesis drug such as growth factors or trophic factors will provide the nutrition the blood vessel development needed. Such artificial environment is similar to real psychological condition and helps heal over the injure tissue.

Although the present invention has been explained in relation to its preferred embodiment, it is to be understood that many other possible modifications and variations can be made without departing from the spirit and scope of the invention as hereinafter claimed.

What is claimed is:

1. A biodegradable thermal-sensitive gel system, comprising at least one polysaccharide solution, at least one electrolytic salt, and at least one weak alkaline solution.

2. The biodegradable thermal-sensitive gel system as claimed in claim 1, wherein said polysaccharide solution is acidic natural polysaccharide solution.

3. The biodegradable thermal-sensitive gel system as claimed in claim 1, wherein at least one polysaccharide solution is selected from the group consisting of chitin, chitosan, glycosaminoglycan, and cellulose.

4. The biodegradable thermal-sensitive gel system as claimed in claim 2, wherein said acidic solution is selected from the group consisting of acetic acid solution, lactic acid solution, citric acid solution, and diluted hydrochloric acid solution.

5. The biodegradable thermal-sensitive gel system as claimed in claim 2, wherein at least one natural protein is selected from the group consisting of gelatin, collagen, fibrin, fibronectin, and elastin.

6. The biodegradable thermal-sensitive gel system as claimed in claim 5, wherein the weight ratio of said natural protein to said polysaccharide ranges between 1:36 w/w and 1:6 w/w.

7. The biodegradable thermal-sensitive gel system as claimed in claim 1, wherein the concentration of said electrolytic salts ranges from 2.8 wt % to 11.2 wt %, and at least one electrolytic salt is selected from the group consisting of glycerol-phosphate, sorbitol-phosphate and glucose-phosphate.

8. The biodegradable thermal-sensitive gel system as claimed in claim 1, wherein at least one weak alkaline solution is selected from the group consisting of sodium bicarbonate, potassium bicarbonate and phosphate buffer saline (PBS).

9. The biodegradable thermal-sensitive gel system as claimed in claim 1, further comprising at least one cross-linking agent selected from the group consisting of glutaldehyde(GA), 1,4-butanediol diglycidyl ether(BDDGE), 1-ethyl-3-(3-dimethyl aminopropyl) carbodiimide(EDC), and Genipin.

10. The biodegradable thermal-sensitive gel system as claimed in claim 9, wherein said cross-linking agent proceeds crosslink reaction in a weak acidic or weak alkaline solution, and the concentration of said cross-linking agent ranges from 0.01 wt % to 0.2 wt %.

11. The biodegradable thermal-sensitive gel system as claimed in claim 1, which is used as a drug matrix.

12. The biodegradable thermal-sensitive gel system as claimed in claim 1, which is used as a drug matrix for angiogenesis.

13. A process for preparing a biodegradable thermal-sensitive gel system, comprising mixing a polysaccharide solution and electrolytic salt to form a mixture, subsequently adding a weak alkaline solution to adjust the pH of said mixture.

14. The process as claimed in claim 13, wherein said polysaccharide solution is an acidic natural polysaccharide solution.

15. The process as claimed in claim 13, wherein at least one polysaccharide solution is selected from the group consisting of chitin, chitosan, glycosaminoglycan, and cellulose.

16. The process as claimed in claim 14, wherein said acidic solution is selected from the group consisting of acetic acid solution, lactic acid solution, citric acid solution, and diluted hydrochloric acid solution.

17. The process as claimed in claim 13, wherein at least one natural protein is selected from the group consisting of gelatin, collagen, fibrin, fibronectin, and elastin.

18. The process as claimed in claim 17, wherein the weight ratio of said natural protein to said polysaccharide is between 1:36 w/w to 1:6 w/w.

19. The process as claimed in claim 13, wherein the concentration of said electrolytic salts ranges from 2.8 wt % to 11.2 wt %, and at least one electrolytic salt is selected from the group consisting of glycerol-phosphate, sorbitol-phosphate and glucose-phosphate.

20. The process as claimed in claim 13, wherein at least one weak alkaline solution is selected from the group consisting of sodium bicarbonate, potassium bicarbonate and phosphate buffer saline (PBS).

21. The process as claimed in claim 13, further comprising at least one cross-linking agent selected from the group consisting of glutaldehyde(GA), 1,4-butanediol diglycidyl ether(BDDGE), 1-ethyl-3-(3-dimethyl aminopropyl) carbodiimide(EDC), and Genipin.

22. The process as claimed in claim 21, wherein said cross-linking agent proceeds crosslink reaction in a weak acidic or weak alkaline solution, and the concentration of said cross-linking agent ranges from 0.01 wt % to 0.2 wt %.

23. A drug delivery system for angiogenesis, comprising a biodegradable thermal-sensitive gel system which contains at least one polysaccharide solution, at least one electrolytic salt, and at least one weak alkaline solution.

24. The drug delivery system for angiogenesis as claimed in claim 23, wherein said polysaccharide solution is acidic natural polysaccharide solution.

25. The drug delivery system for angiogenesis as claimed in claim 23, wherein at least one polysaccharide solution is selected from the group consisting of chitin, chitosan, glycosaminoglycan, and cellulose.

26. The drug delivery system for angiogenesis as claimed in claim 24, wherein said acidic solution is selected from the group consisting of acetic acid solution, lactic acid solution, citric acid solution, and diluted hydrochloric acid solution.

27. The drug delivery system for angiogenesis as claimed in claim 23, further comprising at least one natural protein, wherein the weight ratio of said natural protein to said polysaccharide is between 1:36 w/w to 1:6 w/w, and at least one natural protein is selected from the natural hydrophilic protein group consisting of gelatin, collagen, fibrin, fibronectin, and elastin.

28. The drug delivery system for angiogenesis as claimed in claim 27, wherein the weight ratio of said natural protein to said polysaccharide is between 1:36 w/w to 1:6 w/w.

29. The drug delivery system for angiogenesis as claimed in claim 23, wherein the concentration of said electrolytic salts ranges from 2.8 wt % to 11.2 wt %, and at least one electrolytic salt is selected from the group consisting of glycerol-phosphate, sorbitol-phosphate and glucose-phosphate.

30. The drug delivery system for angiogenesis as claimed in claim 23, wherein at least one weak alkaline solution is selected from the group consisting of sodium bicarbonate, potassium bicarbonate and phosphate buffer saline (PBS).

31. The drug delivery system for angiogenesis as claimed in claim 23, further comprising at least one cross-linking agent selected from the group consisting of glutaldehyde(GA), 1,4-butanediol diglycidyl ether(BDDGE), 1-ethyl-3-(3-dimethyl aminopropyl) carbodiimide(EDC), and Genipin.

32. The drug delivery system for angiogenesis as claimed in claim 31, wherein said cross-linking agent proceeds crosslink reaction in a weak acidic or weak alkaline solution, and the concentration of said cross-linking agent ranges from 0.01 wt % to 0.2 wt %.

33. An angiogenesis method, comprising co-administrating an angiogenesis medicine, at least one polysaccharide solution, at least one electrolytic salt, and at least one weak alkaline solution to the tissue requiring angiogenesis.

34. The angiogenesis method as claimed in claim 33, wherein said polysaccharide solution is acidic natural polysaccharide solution.

35. The angiogenesis method as claimed in claim 33, wherein at least one polysaccharide solution is selected from the group consisting of chitin, chitosan, glycosaminoglycan, and cellulose.

36. The angiogenesis method as claimed in claim 34, wherein said acidic solution is selected from the group consisting of acetic acid solution, lactic acid solution, citric acid solution, and diluted hydrochloric acid solution.

37. The angiogenesis method as claimed in claim 33, further comprising at least one natural protein, wherein the weight ratio of said natural protein to said polysaccharide is between 1:36 w/w to 1:6 w/w, and at least one natural protein is selected from the group consisting of gelatin, collagen, fibrin, fibronectin, and elastin.

38. The angiogenesis method as claimed in claim 33, wherein the concentration of said electrolytic salts ranges from 2.8 wt % to 11.2 wt %, and at least one electrolytic salt is selected from the group consisting of glycerol-phosphate, sorbitol-phosphate and glucose-phosphate.

39. The angiogenesis method as claimed in claim 33, wherein at least one weak alkaline solution is selected from the group consisting of sodium bicarbonate, potassium bicarbonate and phosphate buffer saline (PBS).

40. The angiogenesis method as claimed in claim 33, further comprising at least one cross-linking agent selected from the group consisting of glutaldehyde(GA), 1,4-butanediol diglycidyl ether(BDDGE), 1-ethyl-3-(3-dimethyl aminopropyl) carbodiimide(EDC), and Genipin.

41. The angiogenesis method as claimed in claim 41, wherein said cross-linking agent proceeds crosslink reaction in a weak acidic or weak alkaline solution, and the concentration of said cross-linking agent ranges from 0.01 wt % to 0.2 wt %.
